(19) United States
(12) Patent Application Publication
(10) Pub. No.: US 2004/0057909 A1
(43) Pub. Date: Mar. 25, 2004

(54) Dental polymer film

(76) Inventors: Norbert Moszner, Eschen (DE); Jorg Zimmermann, Lustenau (AT); Volker Rheinberger, Vaduz (LI); Mario Besek, Thalwil (CH); Ulrich Salz, Lindau (DE); Urs-Karl Fischer, Arbon (CH); Patrick Schmidlin, Kappel SO (CH)

Correspondence Address: Nixon Peabody LLP, Clinton Square, P.O.Box 31051, Rochester, NY, 14603-1051, US

(21) Appl. No.: 10/656,465

(22) Filed: Sep. 5, 2003

(30) Foreign Application Priority Data
Sep. 6, 2002 (DE) 102 41 434.3

(51) Int. Cl. A61K 7/16; A61C 5/00

(52) U.S. Cl. 424/49; 433/217.1; 523/120

(57) ABSTRACT

A dental polymer film is described, which is suitable for coating and sealing tooth surfaces, in particular in the approximal space, and characterised in that it contains polymerisable groups.

The present invention relates to a dental polymer film, which can be used for coating tooth surfaces and in particular to protect them against caries.

Caries is a decalcification and dissolving process of enamel and dentine, commencing at predilection sites on the tooth surface and progressing into the depth, caused by bacterial acids and/or accelerated by dietetic acids. Predilection sites include fissures and pits, approximal surfaces in contact with teeth and cervical smooth surfaces. A carious lesion starts initially as an only microscopically visible demineralisation process, wherein the contour surface of the tooth's hard substance area is still retained for the time being. As the carious process progresses further, the still-intact surface layer is finally penetrated. Complete restoration is then no longer possible, among other things due to lack of a matrix structure, to enable re-precipitation of calcium phosphate compounds.

Colonisation with pathogenic germs (plaque) is the main cause of diseases of the hard substance of teeth and of the periodontal apparatus. Streptococcus mutans and lactobacillae are responsible for the caries process, and anaerobes such as Prevotella intermedia, Actinobacillus actinomycetemcomitans, and Porphyromonas gingivalis, among others, for gingivitis and parodontitis.

The sealing of fissures and pits has proved effective as a prophylactic measure. With an absolutely tight, adhesive, polymer-based sealing three aims are achieved: the progression of existing enamel and initial dentine lesions deep down in the fissure is stopped; the sealed enamel becomes insoluble in acid; the caries predilection site is eliminated by changing the morphology of the tooth. However the application of the substance to seal the tooth presents a problem, especially in the approximal space.

For the prevention or therapy of caries and gingivitis, the antimicrobial substances referred to e.g. in S. G. Ciancio, “Agents for the management of plaque and gingivitis”, J. Dent. Res. 71, 1450 (1992) and P. D. Marsh, “Microbiological aspects of the chemical control of plaque and gingivitis”, J. Dent. Res. 71, 1431 (1992) are often used. Examples of these are:

Organic compounds such as e.g. bisbiguanides, chlorhexidine-gluconate or -diacetate, quaternary ammonium compounds such as benzalkonium chloride and cetylpyridinium chloride; phenolic compounds such as triclosan, thymol, hexetidine and inorganic ions such as silver-, zinc-, tin- and copper-ions.

These substances are applied especially in the form of a mouthwash, toothpaste or gel. It is a disadvantage that, due to the relatively short contact time for a clinically successful therapy, application at least twice daily is required. This means that the success of treatment is very strongly dependent upon patient discipline. In addition the active substances used are so non-specific, that non-pathogenic germs are also killed off, and thus the biological equilibrium in the oral cavity is clearly destroyed.

In parodontitis therapy, antibiotics such as, e.g. metronidazole, amoxicillin or doxycycline are also systemically administered in combination with mechanical root polishing. However, with systemic administration, strong side effects are to be expected, which are caused above all by the destruction of the intestinal flora. For this reason, in antibacterial parodontitis therapy, there has been a change over to local application of the active substances in the form of gels, or also by means of coated threads and/or chip-shaped substance carriers, as described in G. Greenstein, A. Polson: “The role of local drug delivery in the management of periodontal diseases: a comprehensive review”; J. Periodontol 69, 507 (1998).

DE 197 43 897 A1, describes agents for the prophylaxis and therapy of approximal caries, which contain a therapeutically effective quantity of a fluoride, which is embedded in a resorbable polymer material, the fluoride being continuously released from the polymer material. Anti-plaque agents or antibiotics are listed as further components. The agent is fixed between the teeth (approximally), where a better hold can in addition be provided with an adhesive layer. The prophylaxis and therapy of approximal caries is thus especially achieved by the release of a fluoride in the approximal cavity. However these agents exhibit only inadequate adhesion to the tooth and, can only with difficulty be completely moulded to the tooth surface in view of their stiffness.

From U.S. Pat. No. 5,626,866 a gel is known as an active-substance-releasing system, said gel being applied to a film and then applied to the mucous membrane or skin. The substance is then released from the gel. The active system can be used in particular as a “nicotine plaster”.

A further solution is described in EP 452 446 B1. In this case, active substances and additives for oral and dental hygiene are processed with a binder to produce a film, and this film is introduced into a patient's mouth. The binder mixture can also be applied to a carrier film. The film is not intended to remain in the mouth for a long period, and therefore only exhibits slight adhesion to the tooth surface.

In summary it can thus be established that the known systems can be applied to the sites to be protected on the tooth surface either only with difficulty, or because of their lack of formability or due to their poor adhesive properties can only serve inadequately to provide complete protection of the tooth surface. Due to inadequate contact, they are also often unable to provide the tooth with any available active substance to a sufficient degree.

The invention is therefore based on the problem of providing a means for coating tooth surfaces and in particular for prevention or treatment of dental caries, in which the abovementioned disadvantages do not occur.

This problem is surprisingly solved by the dental polymer film according to claims 1 to 19. The invention further relates to the use of the polymer film according to claims 20 and 21, the method for coating a tooth surface according to claims 22 and 23, and the kit according to claim 24.

The dental polymer film for coating tooth surfaces according to the invention is distinguished by the fact that the polymer film contains polymerisable groups.

It has surprisingly been shown that the presence of still polymerisable groups in the polymer film leads to a very good formability of the film to tooth surfaces and, following polymerisation of these groups, to cured films with excellent mechanical properties and outstanding adhesion to the tooth surfaces. The film can therefore be used preferably to remain permanently in the approximal region, to which access is particularly difficult.

The providing of the polymerisable groups in the film can be achieved in various ways. For example in the production of the film, the polymerisation can be discontinued at a specific degree of polymerisation, leaving monomers still capable of polymerisation in the film. Also, various monomers with differing reactivities can be used, so that in a first step the more reactive monomers form the film and, in a second step the film applied to the tooth is cured by re-initiation. This can also be achieved by the use of different monomers, which can be polymerised according to different mechanisms, so that e.g. the film is produced by a cationic polymerisation and then cured by a radical reaction. In addition it is possible to treat a polymer film with monomers, i.e. to allow the polymer e.g. to swell in monomers, so that the monomers are absorbed into the film and thus lead to a content of polymerisable groups. Polymers can also be chemically modified, by inserting polymerisable groups.

The introduction of polymerisable groups can be carried out via polymer-analogous reactions. For example, technically accessible synthetic polymers can be used, which are then chemically modified. Examples of these are: polyvinyl alcohol, vinyl alcohol copolymers, e.g. of ethene or vinyl acetate, as well as polyvinyl acetals into which polymerisable methacrylate groups can be introduced by reaction of the available OH-groups with 2-isocyanatoethyl methacrylate or methacrylic acid chloride or anhydride. Likewise any copolymers of acrylic or methacrylic acid can be reacted e.g. with acrylic or methacrylic acid esters, styrene, vinyl acetate, acrylonitrile or acryl- or methacrylamide with glycidyl methacrylate or 2-isocyanatoethyl methacrylate, the polymerisable groups being bound laterally to the primary chain by ester or amide groups. Analogously, chemical modification of biopolymers is also possible. For example cellulose or cellulose derivatives, such as esters or ethers, which are known for their good film-forming properties, can be derivatized via reaction with 2-isocyanatoethyl methacrylate or methacrylic acid-chloride or -anhydride.

It is preferable that the polymerisable groups are at least partially, and in particular predominantly acrylate or methacrylate groups.

The polymerisable films can also be produced by polyaddition of monomers or oligomers or prepolymers, which carry polymerisable groups, in such a manner that at least a part of these groups is available for the further curing of the polymer film.

The dental polymer film according to the invention is preferably based on polyurethanes, Michael-addition resins or other polyadducts.

As polyurethanes are used, in particular the reaction products of diisocyanates, such as e.g. toluylene-diisocyanate, methylene-diphenyl diisocyanate, 2,2,4-trimethyl hexamethylene diisocyanate or isophorone diisocyanate or the oligomeric polyisocyanates produced from these, with suitable polyols, such as ethylene glycol, propylene glycol, glycerol, trimethylolpropane, pentaerythritol, sorbitol, polyethylene glycol, polypropylene glycol or corresponding copolymers or di- or tri-functional polyols produced from these.

Possible catalysts for this reaction are mainly tin-organic compounds or tertiary amines.

For the introduction of the polymerisable groups, the isocyanates are additionally reacted with polymerisable hydroxy compounds, in particular 2-hydroxyethyl(meth)acrylate, hydroxypropyl methacrylate, N-(2-hydroxyethyl)(meth)acrylamide, glycerol mono- and dimethacrylate or Bis-GMA.

By proper selection of the isocyanate- and alcohol components, the flexibility, strength, degree of cross-linking, the swelling properties vis-à-vis different solvents and the hydrophily of the films can be varied both before and after the final polymerisation.

Michael-reaction resins preferably used in the polymer film according to the invention are the reaction products of multi-functional acrylates with di- or multi-functional acetoacetates. Examples of suitable acrylates are ethylene glycol diacrylate, hexane diol diacrylate, tripropylene glycol diacrylate, ethoxylated bisphenol-A-diacrylate, polyethylene glycol-200-diacrylate, trimethylol propane triacrylate, and pentaerythritol tetraacrylate. These acrylates can be reacted in particular with tri- or tetra-functional acetoacetates, such as e.g. trimethylol propane- and glycerol trisacetoacetate and pentaerythritol-tetrakis-acetoacetate, to produce network polymers. As catalysts for the formation of the Michael-addition resins, alkali metal hydroxides, e.g. KOH, tetraalkyl ammonium hydroxides, e.g. tetrabutyl ammonium hydroxide, but in particular bicyclic amidines, such as 1,5-diazabicyclo[4.3.0]-5-nonene or 1,8-diazabicyclo[5.4.0]-7-undecene, and guanidines, especially tetramethyl guanidine, are used. Polyaddition products with non-reacted polymerisable groups are obtained, when the multi-functional acrylates are used in an excess, or acetoacetates with polymerisable groups, such as e.g. 2-acetoacetoxyethyl methacrylate, are used, or solutions of the multi-functional acrylates in mono- or dimethacrylates and/or mixtures thereof are used. By the structure and functionality of the acetoacetates and acrylates, the elasticity, strength and the film-forming properties of the polyadducts obtained can be varied in the desired manner.

Other polyadducts preferably used in the polymer film according to the invention are epoxide-amine adducts, epoxide-anhydride adducts and polysiloxane adducts. Further suitable other polyadducts are accessible via the thiol-ene reaction.

The reactivity and the properties of the polymer film according to the invention can be influenced by use of the following additives in the production of the film.

Radically polymerisable compounds can be used as additives, to increase the strength and E-module of the film, or to reduce the water absorption. The following are particularly suitable for this: (meth)acrylates, styrene and styrene derivatives, allyl compounds or vinyl cyclopropanes, with (meth)acrylates being particularly preferred. Monofunctional monomers are preferred, such as methyl-, ethyl-, butyl-, benzyl-, furfuryl- or phenyl(meth)acrylate, and the multifunctional acrylates or methacrylates known as crosslinking monomers, such as e.g. bisphenol-A-di(meth)acrylate, Bis-GMA (an addition product made from methacrylic acid and bisphenol-A-diglycidyl ether), UDMA (an addition product made from 2-hydroxyethyl methacrylate and 2,2,4-hexamethylene diisocyanate), di-, tri- or tetraethylene glycol-di(meth)acrylate, trimethylol propane tri(meth)acrylate, pentaerythritol-tetra(meth)acrylate, as well as butanediol di(meth)acrylate, glycerol dimethacrylate, 1,10-decanediol di(meth)acrylate or 1,12-dodecanediol di(meth)acrylate are used.

Oligomers and polymers, which carry radically polymerisable groups terminally and/or laterally, can be used as additives, to improve the flexibility of the film and its substrate-adhesion. Particularly suitable for this are α, ω-(meth)acryloyl-terminated polyester-, polyether-, polyepoxy-amine- or polyurethane-telechels or silicic acid polycondensates, such as are obtainable by hydrolytic condensation of silanes. Such silicic acid polycondensates are for example described in DE 44 16 857 C1 or DE 41 33 494 C2. Methacryl- or acryl groups are preferred as radically polymerisable groups.

Cationically polymerisable diluting or crosslinking monomers can be used as additives, to accelerate the curing of the film. The following are particularly suitable for this: glycidyl ethers or cycloaliphatic epoxides, cyclic ketene acetals, vinyl ethers, spiro-orthocarbonates, oxetanes or bicyclic orthoesters. Preferred examples are triethylene glycol divinyl ether, cyclohexane dimethanol-divinyl ether, 2-methylene-1,4,6-trioxaspiro[2.2]nonane, 3,9-dimethylene-1,5,7,11-tetraoxaspiro [5.5] undecane, 2-methylene-1, 3-dioxepane, 2-phenyl-4-methylene-1,3-dioxolane, bisphenol-A-diglycidyl ether, 3,4-epoxy-cyclohexyl methyl-3,4-epoxycyclohexane carboxylate, bis-(3,4-epoxycyclohexyl methyl)adipate, vinyl cyclohexene dioxide, 3-ethyl-3-hydroxymethyl oxetane, 1,10-decane diyl bis (oxymethylene)bis(3-ethyl-oxetane) or 3,3-(4-xylylene dioxy)-bis-(methyl-3-ethyl oxetane).

Cationically polymerisable silicic acid polycondensates can be used as additives to improve the film's hardness and resistance to abrasion. Silicic acid polycondensates are suitable which carry cationically polymerisable groups, preferably e.g. epoxy-, oxetane-, spiroorthoester- or vinyl ether groups. The synthesis of these silicic acid polycondensates can for example be carried out by hydrolytic condensation of silanes, as described e.g. in DE 41 33 494 C2 or U.S. Pat. No. 6,096,903.

Polymer films according to the invention are preferred, in which at least some of the polymerisable groups are radically and/or cationically polymerisable.

For initiation of the polymerisation of the polymerisable groups, the polymer film preferably contains initiators for a thermal, photochemical or redox-induced polymerisation.

These initiators can in particular be present in microencapsulated form. This makes it possible to start the curing reaction by means of pressure or ultrasound.

For initiation of the radical polymerisation, thermal and/or photo-initiators are preferably used. Preferred examples of thermal initiators are the known peroxides, such as e.g. dibenzoyl peroxide, dilauryl peroxide, tert.- butyl peroctoate or tert.-butyl perbenzoate, also in combination with barbitur- or sulphinic acid derivatives. Further thermal initiators are azo-bis-isobutyroethyl ester, azo-bis-isobutyronitril, benzopinakol or 2,2-dimethyl benzopinakol. Examples of suitable photo-initiators are benzophenone, benzoine and their derivatives, or α-diketones or their derivatives such as 9,10-phenanthrenequinone, diacetyl or 4,4-dichlorobenzil. Camphorquinone and 2,2-methoxy-2-phenyl-acetophenone are preferably used, and α-diketones in combination with amines as reducing agents are especially preferably used, such as e.g. 4-(N,N-dimethylamino)-benzoic acid ester, N,N-dimethylaminoethyl methacrylate, N,N-dimethyl-sym.-xylidine or triethanolamine. Acyl phosphines, such as 2,4,6-trimethyl benzoyl diphenyl- or bis(2,6-dichlor-benzoyl)-4-N-propyl phenyl phosphinoxide are especially preferred.

For the curing of cationically polymerisable groups, diaryliodonium- or triaryl sulphonium salts, such as triphenyl-sulphonium hexafluorophosphate or -hexafluoroantimonate are especially preferred, it being advantageous, that these initiators are also suitable for the curing of radically polymerisable groups in the films.

For strengthening, the polymer film according to the invention can contain organic or inorganic fillers. For this purpose these fillers are as a rule incorporated during production of the polymer film. The incorporation of fibre-shaped fillers can be carried out isotropically or anisotropically, depending on application. Preferred inorganic particle-shaped fillers are amorphous spherical materials based on oxides,such as ZrO₂ and TiO₂, or based on mixed oxides of SiO₂, ZrO₂ and/or TiO₂ and especially preferred microfine or nanoparticular fillers, such as silsesquioxanes or metal oxide clusters, pyrogenic silicic acid or precipitation silicic acid with an average particle size of 1 to 500 nm and x-ray- opaque fillers, such as ytterbium trifluoride, barium sulphate, lanthanum or tantalum oxide. In addition however, very short glass fibres, whisker or laminated silicates, e.g. mica particles, can also be used as fillers. It is particularly advantageous for the fillers to be surface-modified using suitable primers.

To prevent premature polymerisation of the polymerisable groups present, the polymer film according to the invention preferably contains suitable polymerisation inhibitors. Aerobic inhibitors are suitable for this, such as aromatic phenols, e.g. hydroquinone monomethyl ether (MEHQ) or 2,5-di-tert.-butyl-4-methyl-phenol (BHT), and in particular anaerobic inhibitors, such as secondary aromatic amines e.g. phenothiazine, N,N′-diphenylphenylene diamine or stable radicals, e.g. the 2,2,6,6-tetramethylpiperidine-1-oxyl-radical (TEMPO).

For stabilisation of the cured polymer film, this can preferably contain one or more antioxidants, e.g. alkylphenols, hydroxyphenyl propionates, hydroxybenzyl compounds or alkylidene-bisphenols and/or light-protection agents, such as UV absorbers of the hydroxybenzophenone- or 2-(2-hydroxyphenyl)-benzotriazol type.

To give the dental polymer film an appearance corresponding to the tooth, it can also contain one or more dyes and/or pigments. Pigments, such as TiO₂ and ZnO, or colour pigments, such as chromophoric xerogels, or dyes such as e.g azo-, phenothiazine- or anthraquinone dyes are especially used. But the use of thermochromic dyes, such as Chromazone Blue (Eckhart), RT31 (Kelly Corp), or pH-sensitive dyes, such as e.g. thymol blue, Congo red, alizarin or phenolphthalein is also possible.

To improve adhesion to the tooth surface, the polymer film according to the invention can preferably have a primer. This can be contained in the polymer film or coated on one side. The dental polymer film is preferably coated on the side facing the tooth surface with a primer for the hard tooth substance. Above all, polymerisable carboxylic acids, phosphonic acids or dihydrogen-phosphoric acid monoesters can be used as primers. Preferred examples are: acrylic acid, methacrylic acid, 4-methacryloyloxyethyloxycarbonyl-phthalic acid (4-MET) or its anhydride (4-META), 10-methacryloyloxydecamethylene-malonic acid, 4-methacryloylaminosalicylic aicd, the phosphoric acid ester of pentaacryloyldipentaerythritol, 2-methacryloyloxydecamethylene-phosphoric acid monoester (MDP), 4-vinylbenzylphosphonic acid or 2-[4-(dihydroxyphosphoryl)-2-oxabutyl] acrylic acid ethyl ester. With such acid monomers, polymer films can also be produced which are capable of ion exchange.

The dental polymer film can in addition have an anti-adhesive additive. The film is preferably coated on the side facing away from the tooth surface with an anti-adhesive additive for plaque, in order to prevent the undesired accumulation of plaque or discoloration. Suitable anti-adhesive additives are fluorinated monomers, such as e.g. perfluorinated alkyl methacrylates.

In addition, the dental polymer film can contain one or more further active substances, the chief possibilities being antimicrobial compounds or antibiotics.

Active substances which can copolymerise via a corresponding functional group in the molecule with the monomers used to produce the polymer film are especially preferred.

Thus they are immobilised by bonding to the polymer, thereby providing the polymer film and in particular its surface with a permanent activity. This leads to adhering germs being killed off, or at least distinctly losing activity. Corresponding polymerisable active substances are described e.g. in EP-A-0-537 774, EP-A-0 705 590 or DE-A-196 54 897.

The antimicrobial active substances which can be used include especially the organic compounds thymol, triclosan, cetylpyridium chloride, chlorhexidine diacetate and inorganic compounds such as zinc chloride and tin fluoride. Metronidazole, minocycline, doxycycline and tetracycline can in particular be used as antibiotics. The active substances are preferably contained in the polymer film according to the invention in a concentration of 0.01 to 10 wt.-%.

Moreover, the polymer film according to the invention can still contain other additives, such as e.g. flavourings, plasticisers, optical brighteners or effect substances.

The polymer film according to the invention can, due to its elasticity and plastic formability, simply be applied to the selected area of a tooth by the user. For simple handling, the polymer film is preferably held in a detachable manner on a carrier film which is in particular translucent, so as not to prevent subsequent photopolymerisation.

In a preferred embodiment, the polymer film according to the invention can also be made up of several layers, the individual layers having special properties. On the one hand, the inner layer, facing the tooth, can be hydrophilic and also have monomers or functionalities and hence be distinguished by a special affinity to the tooth surface, so that there is not even any need to use an additional adhesive for fixing to the tooth surface. On the other hand, the outer layer can, in contrast, be hydrophobic and possess a low plaque affinity.

Due to the flexibility of the not yet cured polymer film, it can be fitted to the curved surface of the tooth in the approximal space without problems. For the better accessibility of the approximal space, a tooth “separator” is used, with which the distance between the teeth concerned can be enlarged and the introduction of the polymer film be facilitated. The polymer film can be adjusted to the necessary size by the user himself, or this can be achieved by using pre-produced film pieces, which preferably have an oval surface. In the subsequent routine procedure, the piece of film is applied directly to the cleaned and/or conditioned tooth surface, temporarily held or fixed with a suitable dental instrument and finally cured in particular by means of light polymerisation.

To support the fit, the polymer film can be applied to the tooth surface using the preferably provided carrier film, and after polymerisation of the polymerisable groups and thus the curing of the polymer film the carrier film is removed.

The polymer film can be fitted to the tooth surface in the approximal space particularly simply using a carrier film, which is formed as an inflatable film bag. During inflation, the resultant “cushion” of carrier film presses the polymer film in the gap optimally onto the surface to be sealed. When using a translucent carrier film, this can remain in the approximal space during light polymerisation. It is not removed until after the desired curing.

It is further possible to fix the polymer film onto the selected tooth surface by means of an adhesive. Such adhesive application can be carried either out by means of the “acid etching technique” or by means of a “self-etching primer”.

In the “acid etching technique”, in a first step with an approx. 35% phosphoric acid solution or a corresponding gel, the dentine and/or enamel surface is conditioned, and then, in a second step a suitable adhesive, such as e.g. the enamel/dentine adhesive Excite® from the company Ivoclar Vivadent AG, is applied to the surface. This pre-treatment leads to a covalent bond finally being achieved between the tooth surface on the one hand and the polymer film applied on the other hand, in that a copolymerisation takes place, in particular of (meth)acrylate groups, which are present both in the adhesive and also in the polymer film.

The particular advantage of the polymer film according to the invention which now becomes apparent, is that due to its flexibility it not only enables the best possible fit to the tooth surface, but due to the reactive i.e. polymerisable groups which it contains, it can also produce covalent bonds to the tooth surface.

If a reaction-adhesion undercoat, i.e. a “self-etching primer” is used, this takes on the function of the phosphoric acid in conditioning the tooth surface. Normally such reaction adhesion undercoats are made in such a way that they contain acid monomers, such as e.g. the polymerisable phosphoric acid- or phosphohic acid methacrylates described in DE 27 11 234. These have an effect on the tooth surface similar to that of phosphoric acid, however they are not rinsed off after the necessary action time, but are polymerised into the adhesive in a second step.

The invention thus also relates to the use of the dental polymer film according to the invention, for coating tooth surfaces and in particular for sealing them and thus for the treatment of or protection from caries. It is particularly preferred that treatment of the tooth surface takes place in the approximal space. The use takes place in particular, in that the polymer film is applied to the tooth surface and fitted to it by shaping, and the polymerisable groups of the polymer film applied and fitted are polymerised.

The invention further provides a method for coating a tooth surface, in which the polymer film is applied to the tooth surface and fitted to it by shaping, and the polymerisable groups of the polymer film applied and fitted are polymerised.

In this case, a method is preferred, in which, for coating of a tooth surface in the approximal space, the approximal space is expanded using a tooth-separator, the surface to be coated is cleaned and surface-conditioned by means of an etching technique, after which an adhesive is applied to the tooth surface thus prepared, and the polymer film according to the invention is introduced into the approximal space and positioned against the tooth surface to be sealed, and its polymerisable groups are polymerised after shaping to the surface, to produce a seal.

In addition the invention relates to a kit which contains the polymer film according to the invention for sealing and mechanical protection of the tooth surface, and an adhesive for fixing the polymer film to the tooth surface.

The invention is described in more detail below, with reference to examples.

### EXAMPLES

In the following examples 1 to 4, the production of the polymer film is carried out by crosslinking Michael addition of pentaerthyritol tetraacetoacetate (PETAA) with either 1,6-hexane-dioldiacrylate (HDDA) or pentaerythritol tetraacrylate (PETA), wherein in each case 1,5-diazabicyclo[4.3.0]-5-nonene (DBN) is used as a catalyst. The following further components were optionally added.

photoinitiator systems, consisting of camphorquinone (CQ) and 4-dimethylaminobenzoic acid ethyl ester (DABE),

photopolymerisable dimethacrylates, such as triethylene glycol dimethacrylate (TEGDMA) or 1,10-decane diol methacrylate (D3MA),

fillers such as Aerosil OX-50 silanised or Aerosil DT-4 (pyrogenic silica, which has been silanised with 3-methacryloyl oxypropyl trimethoxysilane) and/or ytterbium trifluoride (YbF₃).

From the components, in each case two compositions to be mixed together were produced, which were designated basic mixture and catalyst mixture.

### Example 1

The basic mixture and catalyst mixture were produced from the components indicated below with stirring (quantities in wt.-%):

| | Basic Mixture | Catalyst mixture |
|---|---|---|
| PETAA | — | 49.7 |
| HDDA | 47.7 | — |
| DBN | — | 1.1 |
| CQ | 0.6 | — |
| DABE | 1.0 | — |
| TEGDMA | 50.7 | 49.2 |
| | 100.0 | 100.0 |

For the production of the polymer films, basic and catalyst mixtures were mixed, in each case in a 1:1 weight ratio, pressed between two polyester films with 0.1 mm steel bands as spacers and kept at 50° C. for 24 hours to achieve the desired conversion.

After removal of the covering films and spacers, it was possible to cut the polymer films produced to the desired dimensions. The polymer films thus produced were colourless and had no lubrication layer. Subsequent curing with a dental light source (Spectramat®, Ivoclar Vivadent AG) after 2*3 minutes radiation time produced colourless, stable and flexible films.

Measurement of the hardness of the polymer film following Michael reaction and after subsequent curing led to the following result.

| | |
|---|---|
| Film after Michael Reaction | — |
| (24 hours at 50° C.) | |
| Film after second curing | 78 Shore D |
| (2 * 3 mins. Spectramat ®) | |

### Example 2

The basic and catalyst mixture were produced by stirring the liquid components indicated below (quantities in wt.-%):

Aerosil OX-50 silanised and YbF₃ were stirred manually into the basic or catalyst mixture and then homogenised by means of a 3-roller frame (from the company Exakt).

| | Basic mixture | Catalyst mixture |
|---|---|---|
| PETAA | — | 22.7 |
| PETA | 16.9 | — |
| DBN | — | 0.5 |
| CQ | 0.1 | — |
| DABE | 0.2 | — |
| D3MAt | 22.8 | 16.8 |
| Aerosil OX-50 | 40.0 | 40.0 |
| silanised | | |
| YbF₃ | 20.0 | 20.0 |
| | 100.0 | 100.0 |

The production of the polymer films was analogous to that of Example 1.

The polymer films produced with polymerisable groups were opaque and had no lubrication layer. The subsequent photochemical curing led to stable and flexible films. After storage at 37° C. for 72 hours in deionised water, no changes in the film properties could be detected. The water absorption amounted to only 1.85 wt.-%.

Test Result of Hardness Measurement (Average of 5 Measurements)

| Film after Micheal Reaction | — |
|---|---|
| (24 hours at 50° C.) | |
| Film after second curing | 92 Shore D |
| (2 * 3 mins. Spectramat ®) | |
| Storage in water (72 hours at | 91 Shore D |
| 37° C.) and subsequent drying | |
| (24 hours at 37° C.) | |
| Water absorption (in %) | +1.85 |

### Example 3

The basic and catalyst mixtures were produced from the components indicated below:

| | Basic mixture | Catalyst mixture |
|---|---|---|
| PETAA | — | 46.6 |
| PETA | 52.3 | — |
| DBN | — | 1.0 |
| CQ | 0.6 | — |
| DABE | 1.0 | — |
| TEGDMA | 46.1 | 52.4 |
| | 100.0 | 100.0 |

The production and curing of the films were analogous to those of Example 1. Colourless, stable and flexible films were obtained, which had no lubrication layers on the surfaces.

Test Result of Hardness Measurement

| | |
|---|---|
| Film after Michael Reaction | 56 Shore D |
| (24 hours at 50° C.) | |
| Film after second curing | 84 Shore D |
| (2 * 3 mins. Spectramat ®) | |

### Example 4

The basic and catalyst mixtures were produced by stirring the components listed below (quantities in wt.-%) in a manner analogous to Example 2.

| | Basic mixture | Catalyst mixture |
|---|---|---|
| PETAA | 32.6 | — |
| PETA | 36.5 | — |
| DBN | — | 0.8 |
| CQ | — | 0.4 |
| DABE | — | 0.7 |
| D3MA | — | 69.0 |
| Aerosil DT-4 | 30.9 | 29.1 |
| | 100.0 | 100.0 |

The production and curing of the films were analogous to those of Example 2. Opaque, stable and flexible films resulted, whose properties did not change after storage in water.

Test Result of Hardness Measurement (Average of 5 Measurements)

| Film after Micheal Reaction | — |
|---|---|
| (24 hours at 50° C.) | |
| Film after second curing | 90 Shore D |
| (2 * 3 mins. Spectramat ®) | |
| Storage in water (72 hours at | 88 Shore D |
| 37° C.) and subsequent drying | |
| (24 hours at 37° C.) | |
| Water absorption (in %) | +2.05 |

### Example 5

This example shows polymer films based on polyurethanes.

The production of the polymer films was carried out by addition reaction of a triisocyanate to di- and monohydroxy- compounds, which contain further polymerisable acryl- or methacryl- groups. As a catalyst for the formation of the polyurethane, Metatin 802 (di-n-octyl tin diacetate) was used, subsequent curing taking place using a photoinitiator system consisting of camphorquinone (CQ), EMBO ((p-dimethylamino) -benzoic acid ethyl ester) and Lucirin TPO (2,4,6-trimethylbenzoyldiphenylphosphine oxide) and by radiation with a Spectramat®. Aerosil DT-4 was used as filler:

| | |
|---|---|
| GDMA | 22.0 |
| Bis-GMA | 22.0 |
| LS 2294, asymmetric | 35.1 |
| diisocyanate-trimerisate with | |
| iminooxadiazindion structure, | |
| obtainable when starting from | |
| hexamethyl diisocyanate | |
| CQ | 0.1 |
| EMBO | 0.4 |
| Lucirin TPO | 0.5 |
| Aerosil DT-4 | 19.8 |
| Metatin 801 | 0.1 |
| | 100.0 |

For film production, in each case 10 g of the abovementioned composition was mixed with Metatin, and pressed between two Parafilm films with 0.1 steel bands as spacers. The mixture was then left to react at room temperature for 15 hours. The polymer film thus produced was opaque and had no lubrication layer on the surface. Subsequent polymerisation for 2*2 mins. radiation by means of the Spectramat® (Ivoclar Vivadent AG) produced stable and flexible films, which were highly resistant in water.

Test Result of Hardness Measurement (Average of 6 Measurements)

| Film after Michael Reaction | 26 Shore D |
|---|---|
| (24 hours at 50° C.) | |
| Film after second curing | 82 Shore D |
| (2 * 3 mins. Spectramat ®) | |
| Storage in water (72 hours at | 80 Shore D |
| 37° C.) and subsequent drying | |
| (24 hours at 37° C.) | |
| Water absorption (in %) | +1.95 |

### Example 6

To check the adhesive values of polymer films according to the invention to tooth surfaces, the following test set-up was selected:

The polished dentine surface of cows' teeth was etched for 15 seconds with etching gel (37% phosphoric acid gel). The etching gel was then rinsed off and the dentine surface was dried. The dentine adhesive Excite® was massaged onto the so-treated surface for 10 seconds and blown lightly. A film section measuring 4 mm in diameter was laid on the surface. A cylindrical test punch made from polymerised Tetric® Ceram (filling composite made by Ivoclar Vivadent AG) was also thinly coated with Excite® at one end and pressed onto the film. In the last preparation step, the experimental arrangement was irradiated for 60 seconds through the test punch using an Astralis® polymerisation lamp made by the company Ivoclar Vivadent AG. The test pieces produced in this way were then stored in water at 37° C for 24 h. Following storage in water, the adhesion of the film to the dentine was measured with reference to ISO/TS 11405 “Dental Materials—Testing of adhesion to tooth structures”.

Two series of 5 test pieces each were measured, which had been produced using two batches of the polyurethane film according to Example 5.

| | | |
|---|---|---|
| Test series 1: | Average value: | 10.6 MPa |
| | Standard deviation: | 3.1 MPa |
| Test series 2: | Average value: | 11.0 MPa |
| | Standard deviation: | 1.3 MPa |

The fracture sites all showed an adhesive fracture, i.e. the film sections remained undamaged. The adhesion value achieved corresponds to that required of a bracket-adhesive, i.e. an adhesive for fixing tooth brackets, which should show an adhesion of 10 MPa.

1. Dental polymer film for coating tooth surfaces, characterised in that the polymer film comprises polymerisable groups.

2. Dental polymer film according to claim 1, in which at least some of the polymerisable groups are acrylate or methacrylate groups.

3. Dental polymer film according to claim 1 or 2, which is on the basis of polyurethanes, Michael addition resins or other polyadducts.

4. Dental polymer film according to any one of claims 1 to 3, in which at least a part of the polymerisable groups is radically polymerisable.

5. Dental polymer film according to any one of claims 1 to 4, in which at least a part of the polymerisable groups is cationically polymerisable.

6. Dental polymer film according to any one of claims 1 to 5, which comprises initiators for the thermal, photochemical or redox-induced polymerisation of the polymerisable groups.

7. Dental polymer film according to claim 6, which comprises the initiators in micro-encapsulated form.

8. Dental polymer film according to any one of claims 1 to 7, which comprises organic or inorganic fillers.

9. Dental polymer film according to any one of claims 1 to 8, which comprises one or more polymerisation inhibitors.

10. Dental polymer film according to any one of claims 1 to 9, which comprises one or more antioxidants.

11. Dental polymer film according to any one of claims 1 to 10, which comprises one or more dyes and/or pigments.

12. Dental polymer film according to any one of claims 1 to 11, which comprises a primer.

13. Dental polymer film according to any one of claims 1 to 12, wherein the side of the polymer film facing the tooth surface is coated with a primer for the hard tooth substance.

14. Dental polymer film according to any one of claims 1 to 13, which comprises an anti-adhesive additive.

15. Dental polymer film according to any one of claims 1 to 14, wherein the side of the polymer film facing away from the tooth surface is coated with an anti-adhesive additive for plaque.

16. Dental polymer film according to any one of claims 1 to 15, which comprises one or more active substances and in particular one or more antimicrobial compounds and/or one or more antibiotics.

17. Dental polymer film according to any one of claims 1 to 16, which is detachably held on a carrier film.

18. Dental polymer film according to claim 17, wherein the carrier film is in the form of an inflatable film bag.

19. Dental polymer film according to claim 17 or 18, wherein the carrier film is translucent.

20. Use of the dental polymer film according to any one of claims 1 to 19 for the coating of tooth surfaces.

21. Use according to claim 20, wherein
a) the polymer film according to any one of claims 1 to 19 is applied to the tooth surface and fitted thereto by forming, and
b) the polymerisable groups of the polymer film applied and fitted are polymerised.

22. Method for coating a tooth surface, wherein
a) the polymer film according to any one of claims 1 to 19 is applied to the tooth surface and fitted thereto by forming, and
b) the polymerisable groups of the polymer film applied and fitted are polymerised.

23. Method according to claim 22, wherein, for the coating of a tooth surface in the approximal space, the approximal space is expanded by means of a tooth separator, the surface to be coated is cleaned and the surface is conditioned by means of an etching technique, after which an adhesive is applied to the tooth surface thus prepared and the polymerisable polymer film according to any one of claims 1 to 19 is introduced into the approximal space and positioned against the tooth surface and its polymerisable groups are polymerised after forming, to produce a coating.

24. Kit comprising a dental polymer film according to any one of claims 1 to 19 for sealing and mechanical protection of the tooth surface and an adhesive for fixing the polymer film to the tooth surface.
